(19) United States
(12) Patent Application Publication
(10) Pub. No.: US 2010/0274073 A1
(43) Pub. Date: Oct. 28, 2010

(54) Methods and Devices for Predicting Ovulation and Optimum Insemination Time of Animals

(76) Inventor: Philip George Chelf Regas, Aurora, CO (US)

Correspondence Address: James L. Wilcox, Esq.;Joseph Aceto, Esq., 1767 Route 313, Perkasie, PA, 18944, US

(21) Appl. No.: 12/376,305

(22) PCT Filed: Aug. 6, 2007
(86) PCT No.: PCT/US07/17452

§ 371 Date: Feb. 4, 2009

Related U.S. Application Data
(60) Provisional application No. 60/835,799, filed on Aug. 4, 2006.

(51) Int. Cl. A61B 17/43; A61B 10/00

(52) U.S. Cl. 600/35; 600/551

(57) ABSTRACT

Manual techniques for monitoring animal ovulation cycle have been used commercially to determine the best time to breed animals. Techniques using heat as an indicator for ovulation do not always correspond to ovulation. The present invention provides methods, apparatus, and kits for monitoring and predicting ovulation using an automated hand-held device, incorporating a computer and probe for detecting physical changes in the animal body fluid. Changes in electrolyte levels are detected as alterations in resistance which corresponds to specific intervals in the ovulation cycle. The present invention provides a rapid, low cost means for monitoring mammalian ovulation cycles and a tool for use in artificial insemination, especially in assessing large populations of animals.

### CROSS-REFERENCE TO RELATED APPLICATIONS

This application claims priority under 35 U.S.C. §119(e) to U.S. Provisional Application No. 60/835,799, filed Aug. 4, 2006, and PCT/US07/17452, filed Aug. 6, 2007. Each of the aforementioned applications is incorporated in full by reference herein.

### FIELD OF THE INVENTION

The present invention relates to methods and devices for ovulation prediction and monitoring, and in particular to novel instruments and methods useful for the monitoring of ovulation in, and prediction of the optimum time for insemination of, animals and animal fertility monitoring, of, and in particular use for the prediction and confirmation of ovulation in animal breeding applications. The invention further relates to improved animal fertility monitoring, especially of use to breeders and veterinarians.

### BACKGROUND OF THE INVENTION

Many animal breeders rely on manual techniques such as heat indications to determine the best time to breed their animals. Heat can be an indication that ovulation is imminent but it does not always correspond to ovulation. Ovulation can actually take place before, during, or after visible heat. In an animal, after a follicle is selected and released into the fallopian tube, Luteinizing Hormone (LH) allows the egg to be released from the hard follicle. An egg is viable for fertilization for only a few hours once released from the follicle. If the egg is not fertilized within this time, the breeder will have to wait for another cycle. Therefore, the breeder must breed before actual ovulation for conception to occur. As an example, FIG. 1 of the attached drawings illustrates the typical ovulation cycle for horse breeding.

During the course of a normal reproductive cycle in any mammal there are several things that happen prior to ovulation. The first notable and measurable activity is a flood of estrogen into the animal. This precedes a release of LH. The purpose of this hormone is to dissolve the follicle holding the egg. When the follicle is dissolved, the undersized egg is released. The viability of the egg after release can be as few as 8 hours. The ideal scenario for fertilization is having semen present 12 to 24 hours before or as soon after the release of the egg as possible. Having fresh, active semen waiting is ideal. This means that the best time for breeding or inseminating is the day before ovulation or as close to it as possible. Research has shown that if an egg is fertilized later in its maturity, the chances of absorption or abortion are much greater. The percentage of aborted pregnancies in these cases has been reported to be as high as 34%.

Therefore, as can be seen from the foregoing discussion, there is a need for a more accurate methods and devices for the advance prediction of ovulation. This is especially true, by comparison with conventional methods and devices of the prior art, in large herds of animals where optimum fertilization rates are maintained by using conventional techniques of the prior art, with excessive time and costs.

### SUMMARY OF THE INVENTION

The present invention has an objective of overcoming the aforementioned problems inherent in the manual methodologies of the prior art, and provides an ovulation monitor and method for monitoring and predicting ovulation which uses automated technology. The invention is based on automated determination and monitoring of changes in the chemical and electrical characteristics of mucus and saliva, such as occurs in the vagina of an animal, which occur in a predictable pattern from the beginning to the end of the animal's ovulation cycle. It has been established that, as a result of cyclic hormonal changes, there are also changes in electrolytes within the vaginal and cervical mucus of most mammals. By utilizing the automated means provided by the present invention of measuring and recording these electrolyte changes and thereby monitoring them, prediction and confirmation of ovulation to identify the optimum insemination time, i.e., the best time to breed animals, can be accomplished.

Additional aspects of the present invention are provided and are based on improved devices, and methods utilizing the devices, relating to the measuring mediums such as mucus and saliva in various parts of the body of an animal, in addition to vaginal measurements, as well as the attachments/accessories and novel means developed to measure such mediums for the purpose of animal fertility monitoring. Therefore the invention is especially advantageous with regards to the prediction (through the identification of the onset of the ovulatory process, via release of the dominant follicle) and confirmation of ovulation in animal breeding applications.

The improvements over the prior art provided by the present invention are based on novel methods and devices of the invention for providing a unique interpretation by computerized means of electrical resistance readings of mucus or saliva over time in a target mammal. In addition to such interpretation being based on computerized processing of measurements of electrical resistance or conductivity in the vaginal mucus of the target mammal, predictive information is provided on the target mammal's ovulation time course. Additional methodologies are provided by the invention to predict and confirm ovulation in mammals through the measurement of saliva and mucus in other parts of the body, in particular the oral and nasal cavities.

A primary objective of the invention is to determine the optimum time to breed an animal in order to achieve higher conception rates. Although this does not guarantee conception from of a single use (service) of the invention, nor does it guarantee conception in any given animal, it is to be appreciated by those skilled in the art that, when properly used, the invention will reduce the average number of services per conception in nearly all operations, by comparison with conventional methodologies of the prior art. Another objective of the invention is to provide novel means for the prediction and confirmation of ovulation in mammals through the measurement of saliva and mucus in various parts of the body of a mammal, specifically the oral and nasal cavities as well as the vagina. Further objectives and advantages of the invention will be apparent from the disclosure of the invention as set forth herein, the scope of which is intended to be limited solely by the claims appended hereto.

### BRIEF DESCRIPTION OF THE DRAWINGS

FIG. 1 is an illustration of the typical ovulation cycle for a mare.

FIG. 2 shows drawings of small vaginal sensors (a) large vaginal (b) and nasal sensors (c) for use on livestock animals.

FIG. 3 shows the components of the kit described in the present invention.

FIG. 4 schematic view of the mare vagina showing relationship of the cervix with the vaginal mucosa pool.

FIG. 5 drawing of a typical nose ring. The ring contains an electrical resistance sensing device, an internal memory, a power source and a small radio-frequency transmitter for centralized data collection.

FIG. 6 shows screen shots for each of the three main screens; a screen of Data Graph; b. Typical readings with date, reading, and time; c. Typical calculated fertility status for July, August, September.

### DETAILED DESCRIPTION OF THE INVENTION

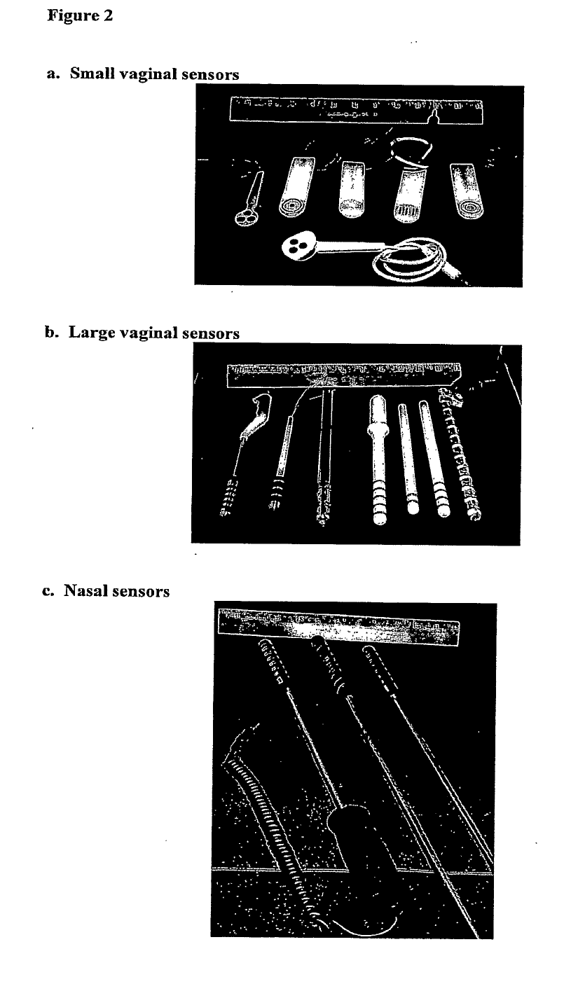

With respect to humans, there has been extensive research and development of oral sensors to detect electrolyte changes in saliva. This has consequently led to the development of products for human use such as, for example, the technology disclosed in U.S. Pat. No. 6,364,844. In animal applications, however, it is less practical to measure saliva due to the difficulties inherent in accessing the target measuring medium, such as saliva in the animal's mouth. Nonetheless, multiple sensors for use in the measurement of electrical resistance in animals have been developed. For example, FIG. 2 shows large/small vaginal and nasal sensors for use on livestock animals of varying sizes.

The existence of such sensors and their use does not guarantee conception in the animal, however, even when used in connection with a single use (a “service”) of the novel methods and devices provided by the present invention, nor does it guarantee conception in any given animal. However, it will be appreciated by those skilled in the art that, when properly used, the present invention will reduce the average number of services that are necessary per conception, in nearly all cases.

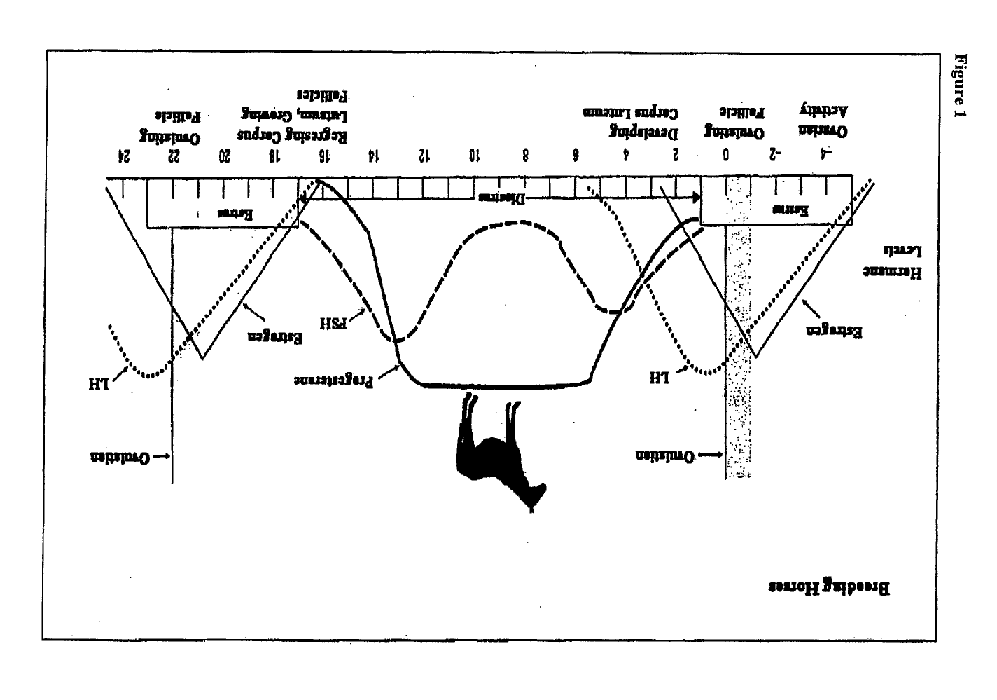

Accordingly, the present invention provides, in a preferred embodiment, an instrument system which utilizes the principles of electricity in an automated manner to identify electrolyte concentrations in an animal. In mammals, hormonal changes during the reproductive cycle (see FIG. 1) have a direct impact on the levels of electrolytes present. During the reproductive cycle, the electrolyte readings of the vaginal mucus will change in direct relation to cyclical changes in hormones like estrogen and progesterone. Just before ovulation, there is a flood of estrogen, which, when the invention is utilized, lowers electrolyte readings that are obtained, and which indicates the onset of ovulation. This is followed by a shift to progesterone dominance, causing a rise in the electrolyte readings and confirming ovulation. The present invention is capable of accurately measuring these changes and of displaying them as a numerical reading and breeding status. Additionally, in its monitoring function, by tracking these readings, the instant invention can effectively identify when ovulation is expected (up to 5 days in advance) as well as confirm when ovulation has occurred. This enables the breeder to have time to plan for insemination to correspond to the lowest reading, which therefore offers the highest likelihood of success. The ease of use of the present invention, combined with a simple daily reading, enables the breeder to identify all of the reproductive cycles of the breeder animals easily. When incorporated into a well-managed breeding program, the invention thus will prove to be an invaluable tool for increasing conception rates.

Using the invention to predict and identify when the breeder animal will ovulate reduces covers, and improves conception rates. The invention accordingly is an essential breeding tool in animal husbandry and for professionals in the field. It is not necessary to be a veterinarian to utilize the advantages provided by the invention; the invention may be used by any knowledgeable breeder.

By charting the readings for each animal in accordance with the invention, the breeder can obtain the advance notice required to inseminate the animal before actual ovulation. In summary, the breeder will want to make sure that fresh active sperm is available to the egg as soon after the release of the egg as possible, but not later than a few hours. This is why, from experience, many breeders breed every other day during heat. This practice can be both wasteful and expensive. The present invention, in contrast, is advantageous over these methodologies of the prior art by enabling tracking of the readings, such that the breeder will accumulate practical information, thereby enabling the breeder to make informed decisions about when to breed the animals.

In the use of the invention, to have the most accurate data, it is important to remove any variables from the reading process, and therefore consistency is important. The more accurate the data, the better the breeding results will be. As the breeder takes and records readings for each individual animal, the breeder must make sure the conditions in which the breeder takes the readings are the same each day. For example, the readings should preferably be taken at the same time each day if possible. This ensures a similar condition and more accurate readings. Readings can vary slightly throughout the day based on activity, diet, urination and other variables.

In a preferred embodiment, the instrument system provided by the present invention includes an ovulation computer utilizing accompanying software employing algorithms in accordance with the invention, as well as a vaginal probe or, alternatively, a device that enables sampling of mucosa, such as a nose clip or similar device that can be inserted or clamped onto or in the nasal cavity of the animal in order to obtain readings in accordance with the invention. In a preferred embodiment, the invention can also include accessories useful in connection with the system such as a carrying case and performance testing fluid to ensure accurate operation of the system. Such probes and sampling devices, and accessories, are well known and commercially available from many sources, and are intended to facilitate the obtaining of readings for the instrument of the invention. For example, for animals that are sensitive to or not accepting of a vaginal probe or sensors, it will generally be preferred to use a nasal clip or nasal mucosa sampling device. It will be appreciated by those skilled in the art that it is the instrument system of the invention that enables the breeder to accurately take a fertility reading and track the fertility status of the breeder's livestock.

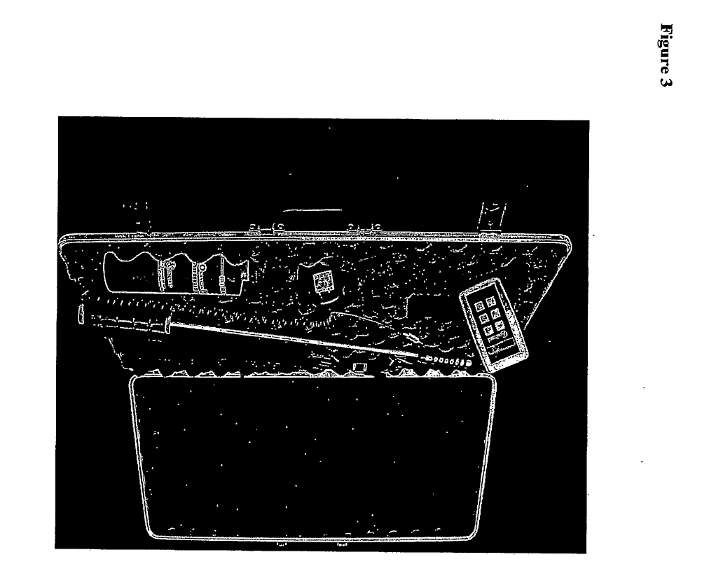

In FIG. 3 of the attached drawings is shown a photograph of a typical kit containing an instrument system and accessories in accordance with the invention:

### Example 1

In a preferred embodiment, the ovulation computer instrument of the invention has a very simple interface for ease of use in the field. On the face of the instrument there are six buttons. On the right side is a plug for the sensor and on the top there is a plug for the download cable into the accompanying software. The following is an example of instructions for use of the instrument:

### ON/OFF Buttons

The ON/OFF buttons are located on the top left and top right of the device. They are labeled as ON/OFF. Push the ON button once to turn the device on. Push the OFF button once to turn the device OFF.

### YES/NO Buttons

The YES/NO buttons are located on the middle of the device, right below the ON/OFF buttons. They are labeled as YES/NO. Push the YES button once to enter the current command request. Push the NO button once to cancel the current command request. Most prompts require a YES keypress to continue.

### NEXT/PREVIOUS Buttons

The NEXT/PREV buttons are located near the bottom of the device, right below the YES/NO buttons. They are labeled as NEXT/PREV. Push the NEXT button to cycle through the menu tree or increase a letter or number. Push the PREV button once to go back to the previous screen or menu item or decrease a letter or number.

### Probe Jack

Plug the sensor or probe cord into this jack on the right side of the unit to take a reading. Make sure the device is off before connecting the probe cord to the jack. Do not bend the cord or stress the jack.

### Download Jack

Plug the download cable into this jack on the top of the unit to take download data to a personal computer. Make sure the device is off before connecting the download cable to the jack. The other will go into the serial port on the personal computer. Do not bend the cord or stress the jack.

### Battery

The invention preferably uses a conventional, commercially available 9-volt battery. This is placed in the back of the unit behind the small cover. Make sure the battery is facing the correct way (see the diagram in the box) and is new. Battery failure will cause a loss of data.

### Menus Available to the User of the Instrument of the Invention:

### Take Quick Reading

This menu option allows the breeder to take a single reading on an animal that will not be stored in memory or associated with any of the animals in the memory of the device according to the invention. This option can be used if the breeder desires to take a quick reading for the breeder's own record, or do a few “practice” readings to check for consistency before an actual reading is logged. Additionally, the breeder can use this menu option if the breeder desires to chart readings manually for additional animals that are not stored in memory, such as if the breeder were using a manual device of the prior art to take readings from such additional animals.

Press ON, then NEXT, then YES to take a quick reading. The device will prompt the breeder to connect the sensor. Connect the sensor and press YES. Then the breeder will be prompted to place the sensor. Insert the sensor and press YES. The instrument will then display the vaginal reading (VR) and the time the reading was taken. Press YES now to return to the main menu.

### Enter Animal IDs

This menu option will allow the breeder to enter IDs to associate with each of the animals the breeder would like to monitor. All animals stored in the memory are referred to by their ID. Up to 5 animal IDs can be entered to be associated with 5 animals that the breeder wishes to breed. Press YES to select this option. Scroll through the list of animals by pressing NEXT. When the breeder wishes to enter or change an ID for an animal press the YES button. Then use the NEXT/PREV buttons to select a letter or number and then press YES to accept it. The breeder presses YES again on a blank space when the breeder is finished defining that animal ID, and either continues to enter animal IDs or presses NO to return to the Main Menu.

### Select Animal

This menu option allows the breeder to choose from the list of animals in the instrument's memory. Animals are selected from the alphanumeric ID associated with each animal. Use the NEXT button to scroll through the list of animals. Press YES to select an animal. Now everything the breeder does will relate to that specific animal, until another animal ID is selected. If the breeder receives the prompt “No animals defined”, the breeder can proceed to the “Enter Animal IDs” section and define the animals first.

### Take Animal Reading

This menu option will take a reading and associate the reading with the current selected animal. To take a reading for a specific animal, the breeder must first select the animal as in the section above. Now just press NEXT to the “Take Animal Reading” prompt and press YES. The invention will prompt the breeder to connect the sensor. Connect the sensor and press YES. Then the breeder will be prompted to place the sensor. Insert the sensor (see below) and press YES. The invention will then display the animal ID and vaginal reading (VR). Press YES now to return to the main menu.

### Calculate Fertility

This menu option will calculate the fertility status of the current selected animal based on the readings for that animal stored in memory. The device will display one of three levels of breed status: “Not ready to breed”, “Breed in 2-4 days”, “Breed NOW.” If there are not enough readings associated with that animal (at least three), the device will display “Not enough readings.” Breed according to the message displayed.

### View, Delete, or Download

This menu option will allow the breeder to review readings of a particular animal, download readings for all animals or a particular animal to the breeder computer, or delete readings for a particular animal. Scroll through these options by pressing NEXT. Select the breeder desired menu option by pressing YES. The following describes the sub-menu options in this prompt in greater detail.

### Review Readings

Press YES to review readings that have already been entered for a particular animal. Press NEXT and then YES to select the animal the breeder would like to review. Press NEXT and PREY to scroll through the readings. Press NO to return to the Main Menu.

### Download Readings

Press YES to this prompt to download readings for a particular animal to the companion software that the breeder has already installed, for example on a Microsoft Windows-based computer. Press NEXT and then YES to select the animal for which the breeder would like to download data. Press YES to confirm the download, connect the cable to the serial port on the breeder's PC (or USB port with the optional adaptor), open the companion software program, select the read device option and press YES. Now press YES to perform the download. The device will return to the View, Delete, Download sub-menu (review readings).

### Download All Readings

Press YES to this prompt to download readings for all animals to the companion software that the breeder will have already installed on a Windows-based computer. Press YES to confirm the download, connect the cable to the serial port on the breeder's PC (or USB port with the optional adaptor), open the companion software program, and select the read device option. Now press YES to perform the download. The device will return to the View, Delete, Download sub-menu (review readings).

### Delete Readings

Press YES to select the delete readings option. Press NEXT and YES to select the animal for which the breeder wishes to delete readings. Press YES to confirm the deletion. The invention will prompt the breeder with “readings Deleted”. Press YES to return to the View, Delete, Download sub-menu (review readings).

NOTE: Although readings are deleted the animal ID will remain. If the breeder wishes to use that position for a different animal, use the Define Animal IDs option to change the ID, otherwise the ID will remain and the breeder will still be able to take readings for that animal.

### Set the Clock

Press YES to select this option that allows the breeder to set the date and time, which is useful for tracking when the breeder animal was checked. Use the NEXT/PREV button to set the correct number. Use YES to accept the setting and move to the next variable (Year, Month, Day, Hour, Minute). Be aware of the A (AM) or P (PM) setting on the clock. The user can press and hold a key to move quickly through the numbers.

### Contact Supplier

This menu option allows the breeder to retrieve the contact phone number and website address for the supplier.

### Setting Up the Device of the Present Invention for Use

### Probe Disinfection

Before the breeder uses the probe associated with the device of the invention, it should be visually inspected for any oxidation (yellowing of the metal) or any other discoloration. If oxidation or discoloration is detected, the probe should be thoroughly cleaned, disinfected and thoroughly rinsed prior to introduction to the breeder animals. Once the entire probe surface is completely cleansed of oxidation, it must be disinfected. Oxidation will not hurt the breeder animal but will affect the breeder readings. “Novasan” or any other similar commercially available disinfectant can be employed. If the breeder is unsure what to use, the breeder veterinarian or veterinary supply outlet to recommend an appropriate disinfectant or contact Animark.

Between animals the breeder will want to clean any mucus from the probe, disinfect and again thoroughly rinse the probe. Disinfectant can irritate the vaginal lining and can also act as a spermicide in the breeder animals, therefore it is important to make sure the probe is 100% clean between readings. See steps below.
1. WIPE the entire surface of the probe from the handle to the tip with a dry paper towel to remove any mucous.
2. WIPE the entire surface of the probe from the handle to the tip with an undiluted disinfectant applied to a moistened paper towel or disposable wipe such as a gauze square or cloth baby diaper. It is not necessary to disinfect the rubber handle of the probe.
3. SOAK the probe by immersing it in a diluted solution of disinfectant made up according to the manufacturer's directions and allow it to stand for at least 30 seconds.
4. RINSE the probe thoroughly with clean running water. This is a critically important step as the disinfectant could irritate or inflame the animal's vagina as well as kill any sperm introduced after the measurement.
5. WIPE down with a dry paper towel.
6. Repeat these steps before testing each animal.
7. Please Note: After each day's use, clean and dry the probe and store the meter and probe in the case. Oxidation may be reduced by storing the probe in the plastic sleeve.

### Example 2

### Using the Invention with Mares

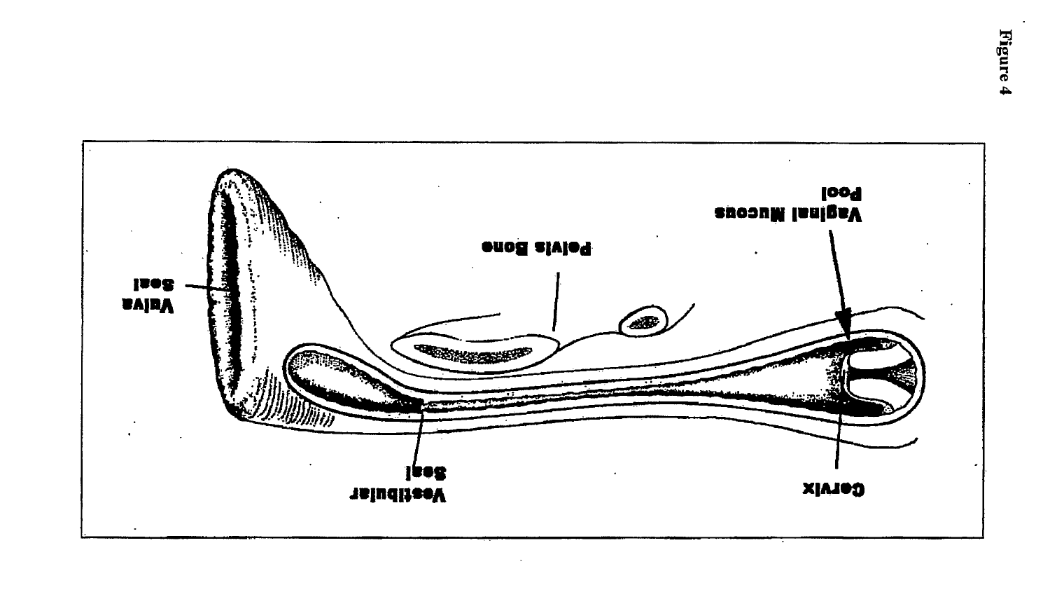

The following example illustrates the use of the present invention in the breeding of mares. In order to effectively predict the onset of ovulation, mares should be checked daily during expected fertile times. To prepare the mare for probe insertion, clean the vulva with soap and water and/or a tamed iodine spray, or as the breeder's veterinarian would recommend in preparation for a specular exam. Insert the disinfected probe between the parted vulva. Take care not to spread the vulva so much that air is allowed to enter the vagina. The introduction of air can alter the accuracy of the readings. Insert the probe at a slightly upward angle to clear the hip skeletal structure, then lift the handle slightly and gently slide the probe into the vagina along the horizontal path until a firm resistance is felt. The probe should be touching the cervix. Raise the handle of the probe slightly upward to drop the tip of the probe into the vaginal pool located just below the cervix (See FIG. 4). Twist the probe for 10-15 seconds to fully coat the sensor portion of the probe with vaginal mucus then hold for a few seconds while the reading is taken (see above for commands to use when taking a reading).

If at least three consecutive daily readings have been taken, use the Calculate Fertility option of the device to determine the breeding status of the mare. Based on the response to Calculate Fertility, do the following:

Not Ready to Breed=>Check again in 12-24 hours

Breed in 2-4 days=>Estrogen release has started, watch carefully for a rise in readings

Breed Now=>Animal is expected to ovulate within 12-24 days, breed as soon as possible

Not Enough readings=>Take at least 3 consecutive daily readings before using this option

### Taking Readings

It is important to take and record readings at approximately the same time each day. Consistency is the key to accurate readings and tracking. Ovulation can occur 1-23 hours after the reading.

Abnormal Conditions (as Indicated by Readings Taken by a Device in Accordance with the Invention)

### 1. High Readings/Air Introduced

Some mares maintain a continuous reading well above the norm, even as high as 400, usually indicating that air is present in the vagina. Whenever this occurs, it prevents good contact between the surface of the probe and mucus in the vaginal tract. If air is accidentally introduced with the probe, it can be manually expelled via rectal palpation or, if a palpator is not readily available, let the animal walk around for an hour or two before trying to take another reading. Normal activity will generally result in the natural expulsion of the air. If the introduction of air continues to be a problem, the breeder may want to try the following procedure: Take a new standard sponge and cut a small hole in it. Make sure the hole is smaller than the probe end. Wet the sponge and place against the vulva. Slowly insert the probe through the hole and into the animal's vaginal tract. This should reduce the amount of air being introduced. In rare occasions, a “wind sucker” that has chronic aerovagina will require treatment before a device in accordance with the invention can be used with good results.

### 2. Ovulatory Disorders

In use, the device in accordance with the invention may not be able to specifically identify the individual disorder present but in general will indicate the there is a potential problem. The breeder's veterinarian should be consulted if the breeder suspects any of these disorders. A veterinarian can usually diagnose and treat any of these rare disorders.
Silent Heat—This is the most common disorder. See “Silent Heat” below.
Anovulation—Heat without an actual ovulation.
Lutenized unruptured follicle—This occurs when the follicle is present and continues to grow but is never released.

### 3. Silent Heat

A “silent heat” mare is usually an animal that does not show any readily observable heat indicators or responses when teased. Using the invention to actually detect when such an animal does ovulate is just as simple as that of any other mare, and readings may be taken every day just as the breeder would for a regular mare. Mares showing no change in readings may have a persistent corpus luteum or a lutenized unruptured follicle. A veterinarian should be consulted if the breeder suspects any abnormal ovulatory conditions other than a silent heat.

### 4. Abnormally Low Readings

Abnormally low readings may be caused by a continuing uterine infection. These readings can also be expected if organic material is being sloughed after parturition during the foal heat.

### 5. Continued Abnormally Low Readings

Continual very low readings can also be caused by urine in the vagina. The urine pooling mare should be treated for this condition before normal conception can be expected, and certainly before normal readings can be taken with device of the invention.

### 6. Twin Ovulation

It is possible for some mares to produce two eggs during the ovulatory cycle. This is the result of the mare ovulating from two ovaries within a two or three day period. In such cases, especially when the mare has a history of twinning, a pregnancy detector or palpation should be used to confirm conception of one or both of the eggs.

### 7. Seasonal Variations

In the mare, there is a tendency for the pre-ovulatory drop to be rather shallow at the beginning of the season and then become much deeper later in the year. For example, when the reading remains consistently around 225, during February and early March, one might see readings drop to only 80 to 120 prior to ovulation. Starting around mid-March until mid-May, they may be seen to drop to a range of 60 to 90, and after about May 15th, they may be seen to be much lower to around 35 to 60. The user may also observe that during the early part of the breeding season, some mares seem to tease warm to hot and will remain in the range of 195 to 120 for many days, sometimes even several weeks. Readings should be continued daily, without breeding, until a reading less than 120 is seen. These specific months do not apply for users in the Southern Hemisphere, where for February it would change to September, etc.

### 8. Introduction of Drugs/Hormones

Introduction of drugs or hormones can and will affect the readings of the device of the invention. If the breeder's veterinarian is introducing drugs or hormones into the breeder animal, the veterinarians' guidance and instructions for breeding should be followed. The device provided by the invention may not show a normal dip pattern as these drugs or hormones tend to by-pass the mare's natural estrogen release pattern and could lead to false indications by the invention.

### Example 3

### Using the Invention with Cows

### The Cow's Cycle

Cows will usually cycle every 21 days. The estrus period and visible heat usually lasts 12-24 hours. The cow's ovulatory cycle can be tracked and ovulation predicted and confirmed by the device and methods provided by the invention just as with other mammals. Most of the time, the hormone that triggers ovulation (luteinizing hormone, or LH) peaks around mid-estrus, and the lowest readings will be found during that time. There is usually a direct correlation with the LH peak. (See FIG. 1). Studies have shown that for the highest conception rates, cows should be bred during this time. Ovulation will usually occur within 24 hours after the LH peak or from the invention's lowest reading.

### Using the Invention for Particular Operations

It is to be appreciated that the invention provides a device that is an improved instrument that is a tool, like any other tool in the breeder's breeding arsenal. The user will need to determine the best time and place to take the readings to ensure maintaining efficiencies with the breeder's standards of operation. The following section is for general suggestions only. For example, the invention can be used as a culling tool if the animal is still ovulating after two cycles. It can also be used to screen for mastitis or ovulatory disorders. Dairy operations will clearly differ from other cattle operations. These recommendations are based on an overall objective of achieving a 12-month calving interval and a higher than average conception.

### Nasal Sensor

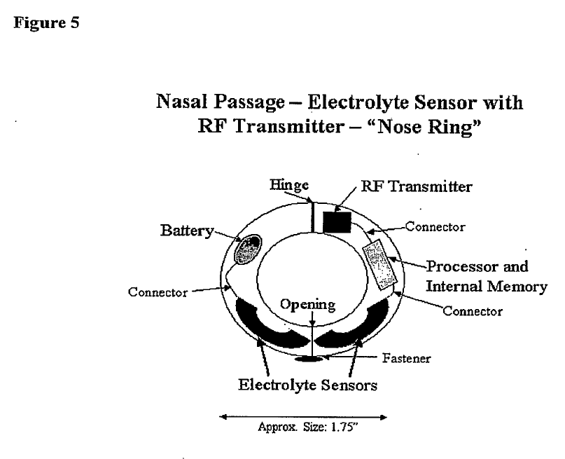

In a preferred embodiment of the invention, a nasal sensor for providing and storing date produced from readings from the nasal mucus of an animal can be utilized to provide data to be analyzed by the device provided by the present invention. It has been determined that one of the most practical locations for the measurement of electrical resistance in bodily mucus in a non-human mammal is in the nasal passages. The embodiment involves the provision of a resistance sensor into a nose ring or other permanently embedded device in the nose of the target animal. The nose ring contains an electrical resistance sensing device, an internal memory, a power source and a small radio-frequency transmitter to send the data to a centralized data collection receiver located at a point near the animal's location or in a causeway or corral into which the animals must pass on a regular basis. FIG. 5 illustrates such a nose ring device in accordance with the present invention.

### Example 4

### Use of the Invention to Provide Nasal Sensor Data

### Data from Nose Ring

The invention can be used with a data collection device such as a nose ring containing conventional radio frequency transmitter means that transmits the data from the invention to a radio frequency receiver. Data points collected and stored in the internal memory of the nose ring are defined as follows:

Data Definition Table

| Field | | Type | Length |
|---|---|---|---|
| 1. | unique animal ID | Alphanumeric | 8 chars |
| 2. | date of electrical resistance reading | Date | 8 chars |
| 3. | time of electrical resistance reading | Time (military) | 4 chars |
| 4. | electrical resistance of the medium | Numeric | 3 chars, |
| | defined on an arbitrary calibrated scale | | |
| 5. | temperature of the animal | Numeric | 3 chars |
| 6. | date of last data download | Date | 8 chars |
| 7. | time of last data download | Time (Military) | 4 chars |

The radio frequency receiver, which can be an external RFID data receiver which is commercially available such as the PGS RFID Reader from PGS Systems, then collects the data at a predetermined interval (usually daily), depending on the perimeter access to the transmitter, and stores it on a central computer or PC for analysis and determination of the breeding and general health status of the animal. In particular the collected data can be used to determine the onset of ovulation via the determination of the timing of the release of the dominant follicle in the ovary.

### 1. Cows Detected in Standing Heat

On average, 50%-60% of cycling cows are detected in heat using traditional methods. Provided an adequate first service conception rate is being achieved, these cows may be bred in the usual manner. However, if the breeder determines that a higher conception rate is desirable, the following procedure may be useful:
a) Take a reading from a device in accordance with the invention soon after the cow is first observed in heat.
b) Take a second reading therefrom after 6-12 hours.
   If reading is similar (+ or −10) to first reading, then breed now.
   If reading is lower by 10 or more, take an additional reading in 4-6 hours. Breed when reading is similar to or higher than previous reading.
   If reading is higher by more than 10, cow is ovulating early and should be bred immediately. If cow returns to estrus, remember to breed earlier than usual at the following estrus.
c) Follow post-breeding, check with an ultrasound scanner, such as the commercially available “Pregscan” external ultrasound.

### 2. Cows not Detected in Heat

Cows not detected in heat by 40-50 days post-calving should be monitored by the breeder using the device of the invention. Cows in the luteal phase of the cycle (usually from the third to sixteenth day of the cycle) will give relatively high invention readings (90 or greater). Monitor these cows as follows:
a) Take readings every other day.
b) Readings will begin to decline 2-3 days prior to estrus. Take readings daily at this stage.
c) When readings have declined to 50-60, cows must be probed at least morning and evening, or preferably every 6-8 hours.
d) When a reading does not show a decline from the previous value, or is slightly higher, the cow should be inseminated.
e) Follow post-breeding check with a pregnancy test (commercially available)

### 3. Post-Breeding Check, Late Ovulation

Late or delayed ovulation: A reading should be taken 24 hours after breeding. Cows ovulating normally will show a reading higher than the one taken at time of insemination. However, late ovulators will usually have a reading similar to that at the time of breeding. If a high probability of conception is desired, the breeder may elect for a second breeding of these cows at this time.

### Example 5

### Testing the Accuracy of Readings Taken by the Device of the Invention

To test the device provided by the invention and ensure the readings the breeder is taking each day therefrom are accurate, the breeder may wish to set up a performance test liquid. To do this, simply take the performance test liquid container, which is preferably included in the accessories accompanying the device of the invention and fill it with tap water. Insert the probe into the container and take a Quick Reading. Then take a pinch of salt and add it to the water. Take another Quick Reading with the probe and note the reading. When salt is introduced into the tap water, the readings on the breeder invention should drop. If each time salt is introduced the probe takes a lower reading, the instrument is working correctly. If the readings do not drop, check the probe for oxidation. If oxidation is detected then clean and disinfect the probe as per conventional disinfection guidelines well know in the industry.

As described above, the present invention also provides novel algorithms for performing the methods of the invention, embodied in software for use in conjunction with the device of the invention, which when used properly in combination advantageously provide an efficient electronic method of detecting and predicting ovulation, that has been shown to be reliable in large numbers of animals. Because consistency is the key factor in the breeder's continued success, missing days and spotty testing will not likely provide the breeder with the level of success the breeder is looking for. The software gives the user a central area to store readings and the ability to analyze those readings to determine the best time to breed animals. Properly used, the practice of these methods of the present invention will provide a high degree of information on the breeder animal's ovulation cycle.

The following is an example of a preferred embodiment of operation of the methods of the invention employing the novel algorithms embodied in software resident in the device of the present invention. The example is not intended to limit the invention in any way, but is presented for illustrative purposes of the use of the novel software provided by the invention in conjunction with the device of the invention.

The software, in a preferred embodiment, is designed to function on a Microsoft Windows compatible computer loaded with Windows XP, 2000, NT or newer, a CD-ROM drive and a least 5 Megabytes (MB) of free space. To install the software, the user loads the software onto a personal computer, and after the software is installed and running on the computer, the following menu options are available to the user:

1. Select Animal
This option allows the breeder to select what animal the breeder will be working with until the breeder selects another animal. Select the animal from the list by clicking on it and press OK, or if it does not exist, the breeder must create it first on the ovulation computer of the invention and then download readings to the software on the computer to access that animal. Alternatively, the breeder can create a data file without a computer download, by opening, for example, in Windows Notepad (in accessories) and then save the file in the DATA folder within computer folder as: XXXX.dat, where XXXX is the name of the animal or reference ID.

2. Read the Device to Monitor Ovulation
To download readings from the device “ovulation computer” to enable monitoring of ovulation, the breeder must first connect the ovulation computer to the breeder's personal computer by plugging in the download cable into the top of the ovulation computer and the other end into a serial port on the breeder's computer. Once the ovulation computer's download cable is connected, select the “Read ovulation computer” option and press OK to the port selection. If the breeder does not know the number of the breeder's serial (COM) port, use 1 or 2. If the breeder is using the USB adaptor, follow the manufacturer's directions for selecting the port number. If the breeder receives an error message that the port is being used by another program, exit out of all other software and try again. At this point, the breeder will see the “Waiting for ovulation computer” prompt and a blank progress bar.
Now on the ovulation computer monitor, select the download option for all animals or just the animal the breeder currently have selected. The ovulation computer will prompt the breeder to open the download software (which the breeder has already done), so select YES. Select YES again to the Ready to download prompt and watch the progress bar on the breeder's computer. It should turn red for a second or two as the download completes. Now the breeder's new data is available for selection as in option 1.

3. View Readings
This option allows the breeder to view the readings stored for the currently selected animal. The format of the readings is the date (month, day, year), then the actual reading and the time the reading was taken. The user can now move up and down in the data view or print or delete readings. If the breeder selects PRINT, the readings will be printed to the breeder's default printer. If the breeder selects DELETE, the breeder will be prompted to confirm the deletion, as once the readings are deleted, they are permanently removed.
Note: The user can open the data readings in a spreadsheet program if the breeder needs further data manipulation capabilities. To do so import it as a Tab delimited file and label columns as needed.

4. Help
This option provides the contact information to reach the commercial supplier of the invention.

5. Exit
Use this option to exit from the software. The program will automatically save the data into the data folder in the directory.

5. Calculate Fertility
The Calculate Fertility option is the most important option in the software. What this option does is interpret the data and identify on a calendar what day the breeder should breed the animals. There are three key responses to this option:
   Not Ready to Breed—this means the animal is not going to ovulate any time soon and additional daily readings should be taken.
   Breed in 2-4 Days—this means that the animal has started the ovulation process (selected a dominant follicle) and will normally ovulate within 2-4 days.
   Breed Now—this means that the animal will ovulate within 12-24 hours and breeding should occur as soon as possible.

These three responses are color coded on the calendar for ease of use. When viewing readings, make sure the breeder is looking at the right date of the readings by using the large blue arrows to move forward and backwards through the data. The date is displayed across the top of the window.

When finished viewing data in this window, select the exit button.
Note: The user can have the main window, the View Readings window and the Calculate Fertility window open at the same time. Just move them (click and drag) to different parts of the screen for full viewing capabilities.

6. Capacity
The software can hold as much data and as many animals as the breeder have room for on the breeder's computeer hard drive—there is no practical limit. This allows the breeder to have a historical record of each animal so the breeder know the likely hormonal and ovulation computer reading pattern the next time the animal is to be bred.
   An example of menu operation showing the basic functionality of the software of the invention, the operation of which is described above, is set forth below:

```
Main Menu
    |
    -- Take Quick Reading → connect sensor → sensor in
    place? → “VR=xxx”
    |
    --- Enter Animal IDs → Select ID → NEXT/PREV for
    letters → YES/YES to exit
    |
    -- Select Animal → NEXT/PREV → main
    |
    -- Take Animal Reading → connect sensor → sensor in
    place? → “VR = xxx”
    |
```

| | |
|---|---|
| -- Calculate Fertility | → “Not enough readings” |
| | → “Not ready to breed” |
| | → “Breed in 2 - 4 days” |
| | → “Breed Now” |

```
    |
    ---View, Delete, Download
    |
        -- Review readings → select ID → NEXT/PREV
        |
        -- Download Readings → select Animal ID → Open
            Software → Ready to download? → “Downloading”
        |
        -- Download All Readings → Open Software → Ready to
            download? → “Downloading”
        |
        -- Delete Readings → select ID → confirm → delete
            Animal ID? → animal deleted →
    |
```

### Further Examples Illustrating the Invention

### 1. Permanent Sensor/Transmitter/Receiver/Software

A further example of an implementation of a preferred embodiment of the methods provided by the invention, in an automated breeding monitor, is based on the collection of electrical resistance data from the vaginal mucus of the animal by means of the placement of a sensor in the vaginal tract. The device then stores the reading data (date, time, reading) in an internal memory, which is then downloaded to a centralized location periodically (usually within days) via a download cable into a personal computer.

### Temporarily Placed Sensor

In a still further preferred embodiment of the present invention, by combining the sensing and data transmittal into one device, as in the nose ring described above, devices in accordance with the invention can collect electrical resistance data independently of an intermittent device (the sensor), which must be placed each day, thus leading to greater productivity, safety and accuracy for the user.

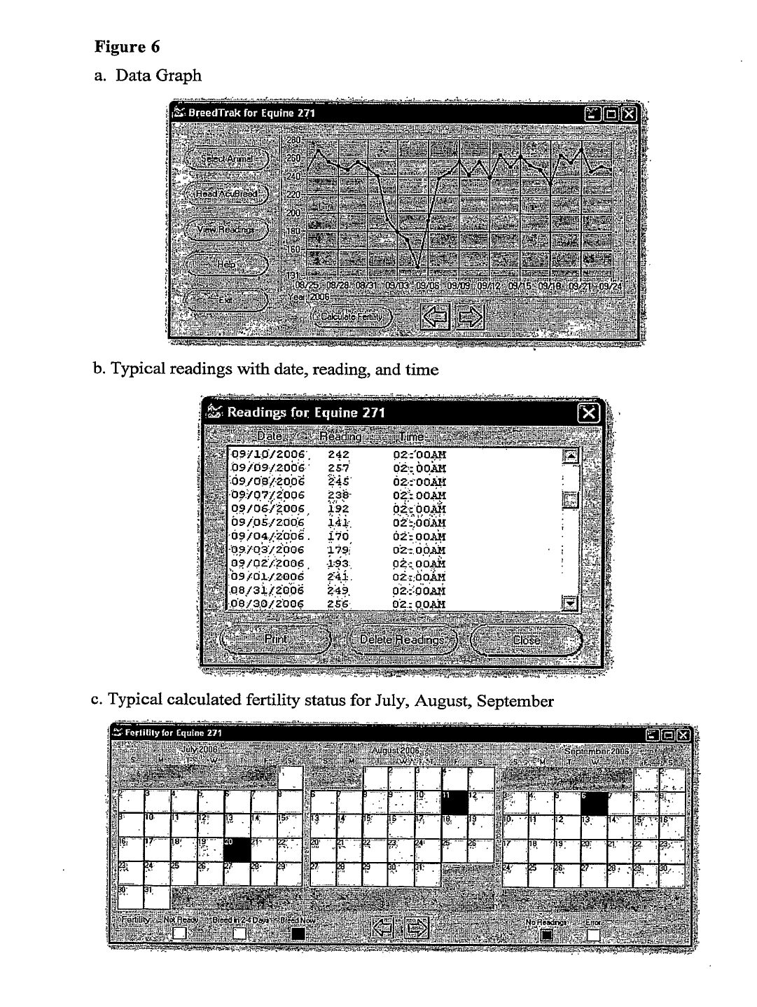

It is to be appreciated that the novel methods provided by the invention and implemented in software have been developed to provide centralized analysis and interpretation of the data collected, independently of the data collection method (for example, saliva, nasal or vaginal mucus) and device (for example, external or internal, implanted or nose ring). It is important to note that generally, this software enables the storage of data in a large population of animals and has, as a data limit, only the capabilities of the computer in which it is operating. For example, the specific software can be written so that it is capable of providing a graph of the data, presenting it in a list format for printing and data review, and on a calendar, such that breeding status is shown by color code where increasingly darker shades of color on each day of the calendar indicate increasing fertility of the animal being analyzed. Access to an individual animal's data can be made through the definition of an animal ID made up of alphanumeric characters, defined at the option of the user. Additional data for use with the software can be defined as in the data definition table above. Screen shots showing each of the three main screens of one example of software implementation in accordance with the teachings of the invention, are set forth in FIG. 6 of the attached drawings.

The following is an example describing a further preferred embodiment of software (designated “BreedTrak”) which is intended to be used with a device (designated “AcuBreed”) in accordance with the invention.

The software, designed to run on any IBM PC compatible machine, running, for example, Microsoft Windows software.

### Installation of Software

a. To begin the installation process, insert the CD into the CD-ROM drive. Using Explorer or My Computer, open the CD-Rom drive and double click on the file: breedtrak.msi. This will start the install program.
b. You will then be prompted to enter the directory into which you want to install the software. If you do not have a reason to change the directory, accept the default of c:\program files\breedtrak.
c. The progress bar will then update you on the status of the installation. When the install is complete, you will see a message: “BreedTrak Installation Successful”, select Finish. This will restart your computer.
d. If the program does not install correctly, go back to the Windows desktop, ensure all other programs are closed and try the installation again.
e. If you need to uninstall the program for any reason, just open the BreedTrak folder and double click on “uninst.exe”. This will uninstall the program and delete the BreedTrak program files.

### Running the Program

1. To run the program, double-click on the BreedTrak program icon on your desktop, or select BreedTrak from your Start menu.
2. The first time you run the program, you will be prompted to enter an Animal ID. If you are downloading data for a specific ID, enter that ID. If you are downloading data for All ID's, just press okay and BreedTrak will automatically set up an ID for each ID stored in the AcuBreed.
3. To exit the program, just select the “Exit” option in the lower left corner of the BreedTrak program window.

### Downloading Data

1. Once you have readings stored in your AcuBreed you can download them into BreedTrak for charting, viewing and printing. To download readings into BreedTrak, select the “Read AcuBreed” option located on the upper left hand corner of the BreedTrak window.
2. Note: If you receive an error: “COM port problem,” you must exit out of BreedTrak and then run whatever program is reserving the COM port at the time (for example, Palm Pilot HotSync®), and then exit out of that software completely.
3. Now, connect the BreedTrak download cable to the jack at the top of the device and the serial port on the back of the computer. There is usually only one serial port on each computer to plug into. If you have don't know where the serial port is, just match the cable plug to the computer port. If you don't have a serial port, please let us know and we can provide a serial port adaptor for the USB port.

4. Select the COM port you are using on the Download Readings window (usually COM1). On the AcuBreed, go into the “Download Readings” prompt and select the ID you wish to download for or select “Download All Readings”. Select “YES” to the AcuBreed prompt, “Ready to Download?” When you do this, watch the progress bar on the computer, which should fill in with red as the readings download. This could happen very fast, so try not to miss it.
Note: You can download to BreedTrak as often as you like. The new readings will always be added to the previously stored readings.

### BreedTrak Main Window

1. After you have downloaded readings, you will see them displayed on the graph on the main BreedTrak window.
2. Use the big blue arrows on the bottom of the window to move back and forth through your data. Use the left arrow to move to earlier dates, and the right arrow to move to later dates.
Note: BreedTrak will automatically select and adjust the dates and scale of your graph to match your data.
Note: you cannot easily change data in BreedTrak. This is to prevent errors and keep the consistency and accuracy of the data at the highest possible levels. If you must change the data, use Notepad and the instructions below.

### View Readings

1. The “View Readings” option will bring up a new window that will display all AcuBreed data that you have downloaded to the BreedTrak program.
2. The format of the data shows the date the reading was taken, and time the reading was taken.
3. Use the arrow keys on the right side of the window to move up and down through your readings.
4. Select the “Print” option to print a copy of the data in your BreedTrak window. This will print in a basic text format. To print graphs you must convert the data to another program (see section on Exporting Data)
5. If you would like to delete all your data from the BreedTrak file, select the “Delete” option. As there is no way to reverse this process once selected, you will be prompted to confirm your choice.
6. To exit out of the View Readings window, select the “Close” option.

### Calculate Fertility

1. Select the “Calculate Fertility” option on the bottom of the screen to see your fertility status plotted on a calendar. This will display your fertility status for each day with darker shades of blue representing increasing fertility.
2. Note: days without data will be shown as a blank gray square. Days that are marked as yellow represent an error in the data (not what BreedTrak was expecting). In this case, focus on the dates you are attempting to breed or you can contact the supplier for more information.
3. Each day in the calendar shows the date and the fertility status for each day. Use the big blue arrows on the bottom of the screen to move back and forth through your data. Use the left arrow to move to earlier dates, and the right arrow to move to later dates.
4. To close the “Calculate Fertility” window, just click on the “X” in the upper right hand corner of the window.
5. Note: “Calculate Fertility” displays in a separate window. You can move this window on your screen to view your graph and calendar at the same time.

### Other Options

1. The “Help” option displays the contact information of the supplier. The “Exit” option is used to close the BreedTrak window. All data will be automatically saved and the program will close and return you to your last open window.

### Changing or Exporting Data

1. If for some other reason you must edit your data manually, you can use Microsoft Notepad or a similar text-only editor to add or change your readings.
2. To change your readings, open the file “Readings.dat” in the BreedTrak directory. Add or change readings as needed. Be sure not to add any extra characters than necessary or change the format of the file in any way.
3. Save the file as text only with the name: readings.dat and exit the editor program.
4. Note: please refer to the file “readme.txt” in the BreedTrak directory for more information on the readings.dat file format.
5. If you would like to print a graph or change your data format, you can import the data into Microsoft Excel or similar program. To do so, open Excel and then open the file “Readings.dat” in the BreedTrak directory.
6. Select “delimited with tabs” as the file format. You can usually leave the column formats as they appear in the import. If you have problems with the data formats, you can still change them after the data is imported.
7. If you have difficulty importing, do not save the file, but try the import again.
   Important: do not save the changed file as readings.dat, as this will render it unusable by BreedTrak. Instead choose a different name for the imported file, such as importdata.dat, and save it to the BreedTrak directory.

While certain of the preferred embodiments of the present invention have been described and specifically exemplified above, it is not intended that the invention be limited to such embodiments. Various modifications may be made thereto without departing from the spirit of the present invention, the full scope of which is delineated in the following claims.

1. An automated method for artificial insemination in a target mammal comprising:
a. sampling a source of body fluid in a target mammal;
b. determining physical parameters in said source, said parameters are from a group consisting of chemical, electrical, and combinations thereof; and
c. analyzing said parameters during said target mammal ovulation cycle.

2. The method of claim 1 wherein said body fluid is from a group consisting of nasal fluid, mucus, saliva, and combinations thereof.

3. The method of claim 2 wherein said source is from a group consisting of nose, mouth, vagina, and combinations thereof.

4. The method of claim 1 wherein said physical parameter is from a group consisting of resistance, conductance, and combinations thereof.

5. The method of claim 1 wherein said analyzing is with an ovulation computer instrument and probe.

6. The method of claim 1 wherein said analyzing assesses the fertility status of said target mammal.

7. The method of claim 1 wherein said target mammal is a mare.

8. The method of claim 7 wherein said sampling is daily.

9. The method of claim 8 wherein said sampling comprises:
a. cleaning the vulva;
b. inserting said probe to cervix;
c. lowering tip of said probe into vaginal pool; and
d. coating said tip with said mucus.

10. The method of claim 1 wherein said target mammal is a cow.

11. A automated instrument system for determining the artificial insemination period in a target mammal comprising:
a. an ovulation computer to process information obtained during said target mammal ovulation cycle;
b. an algorithm to provide an optimum time course for insemination based on physical parameters from said target mammal body fluid;
c. a probe wherein said probe effects sensors for detecting said target mammal body fluid; and
d. a power source.

12. The system of claim 11 wherein said computer stores information obtained from multiple target mammals.

13. The system of claim 11 wherein said probe is obtained from a group consisting of a vaginal probe, a nose clip, nasal mucosa sampling device, and combinations thereof.

14. The system of claim 11 wherein said power source is a 9 volt battery.

15. A kit for monitoring the ovulation cycle in a target mammal comprising:
a. an ovulation computer;
b. software;
c. probe;
d. performance testing fluid; and
e. a carrying case containing a, b, c, and d.
